(19) United States
(12) Patent Application Publication
(10) Pub. No.: US 2026/0240650 A1
(43) Pub. Date: Aug. 20, 2026

(54) TRANSLUMINAL DELIVERY SYSTEM

(71) Applicant: CARDIOVALVE LTD., Or Yehuda (IL)

(72) Inventors: Michael ALBITOV, Kiryat Ono (IL); Maxim KARALNIK, Karmiel (IL); Meni IAMBERGER, Kfar Saba (IL); Ilia HARITON, Zichron Yaakov (IL); Tal HAMMER, Ramat Gan (IL)

(73) Assignee: CARDIOVALVE LTD., Or Yehuda (IL)

(21) Appl. No.: 18/869,929

(22) PCT Filed: Jun. 7, 2023
(86) PCT No.: PCT/IL2023/050586

§ 371 Date: Nov. 27, 2024

Related U.S. Application Data
(60) Provisional application No. 63/350,200, filed on Jun. 8, 2022.

(51) Int. Cl. A61F 2/24

(52) U.S. Cl. CPC A61F 2/2436

(57) ABSTRACT

Apparatus including a delivery tool dimensioned for percutaneous delivery of an implant (20) to a heart, which includes: (a) a catheter (132) defining a catheter-lumen (133), (b) a shaft (1034), at least a portion of which is slidably housed within catheter-lumen (133), and (c) a distal portion (304) that defines a distal portion axis. Distal portion (304) further includes: (i) a capsule (1064) able to transition between a first state in which capsule (1064) is disposed at a first distance from a distal end of catheter (132), to a second state in which capsule (1064) is disposed at a second greater distance from the distal end of catheter (132); and (ii) a support (300), configured to expand, while capsule (1064) transitions from the first state to the second state. Other embodiments are also described.

### CROSS-REFERENCE TO RELATED APPLICATIONS

The Present Application Claims the Priority of U.S. Provisional Patent Application 63/350,200 to Albitov et al., entitled, “Transluminal delivery system,” filed Jun. 8, 2022.

The present application is related to International application PCT/IL 2021/051433, filed Dec. 2, 2021, and entitled “TRANSLUMINAL DELIVERY SYSTEM,” which published as WO 2022/118316, and which claims priority from and is a Continuation in Part of U.S. application Ser. No. 17/399,594, filed Aug. 11, 2021, entitled “TRANSLUMINAL DELIVERY SYSTEM,” which published as US 2022/0175526, which claims priority from U.S. Provisional Application 63/120,808, filed Dec. 3, 2020, entitled, “TRANSLUMINAL DELIVERY SYSTEM.”

Each of the applications mentioned above is assigned to the assignee of the present application and is incorporated herein by reference.

### FIELD OF THE INVENTION

Some applications of the present invention relate in general to transluminal implant-delivery systems. More specifically, some applications of the present invention relate to prosthetic heart valves, and transluminal delivery systems therefor.

### BACKGROUND

Dilation of the annulus of a heart valve, such as that caused by ischemic heart disease, prevents the valve leaflets from fully coapting when the valve is closed. Regurgitation of blood from the ventricle into the atrium results in increased total stroke volume and decreased cardiac output, and ultimate weakening of the ventricle secondary to a volume overload and a pressure overload of the atrium.

### SUMMARY OF THE INVENTION

Applications of the present invention are directed to apparatus and methods for delivering an implant to a subject.

For some applications, there is provided, in accordance with an application of the present invention, apparatus for use at a heart of a subject, the apparatus including:
a delivery tool dimensioned for percutaneous delivery of an implant to the heart, the delivery tool including:
   a catheter defining a catheter-lumen,
   a shaft, at least a portion of the shaft slidably housed within the catheter-lumen, and
   a distal portion that defines a distal portion axis and includes:
      a capsule, the capsule:
         configured to house at least a portion of the implant,
         coupled to a distal portion of the shaft, and
         slidable along the distal portion axis, with respect to the catheter, so that the capsule transitions from:
         a first state in which the capsule is disposed at a first distance from a distal end of the catheter, to
         a second state in which the capsule is disposed at a second distance from the distal end of the catheter,
         the second distance is greater than the first distance; and
      a support, the support:
         fixedly coupled to a distal region of the catheter,
         extending:
         along the distal portion axis, and
         from the distal end of the catheter toward the capsule, and
         configured to expand, while the capsule transitions from the first state to the second state:
         from a contracted state in which the support has a first length, to
         an expanded state in which the support has a second length.

In an application, a proximal region of the support is fixedly coupled to the distal region of the catheter.

In an application:
the support is configured to define a support-lumen,
a portion of the shaft is disposed within the support-lumen, and
while the support is in the expanded state, the support has a stiffness that is greater than a stiffness of the portion of the shaft.

In an application, the support is configured such that while the capsule transitions from the first state to the second state, the support self-expands from the contracted state to the expanded state.

In an application:
the capsule is reversibly slidable along the distal portion axis, with respect to the catheter, such that the capsule is transitionable from the second state to the first state, and
while the capsule transitions from the second state to the first state, the support is configured to contract from the expanded state to the contracted state.

In an application, while the support is in the contracted state, the support has a radial thickness of 1-3 mm.

In an application, while the support is in the contracted state, the support has a length of 4-18 mm.

In an application, while the support is in the expanded state, the support has an expanded length that is between 10 mm and 60 mm.

In an application:
while the support is in the contracted state, the support has a contracted length, and
the expanded length that is 10-50 mm longer than the contracted length.

In an application:
while the support is in the contracted state, the support has a contracted length, and
the expanded length is 2-5 times longer than the contracted length.

In an application, the support is configured such that while the support is in the expanded state, the support extends from the distal end of the catheter to the capsule.

In an application, the support is configured such that while the support expands from the contracted state to the expanded state, the support extends from the distal end of the catheter to the capsule.

In an application:
the support includes a plurality of coaxial segments,
a proximal one of the coaxial segments is coupled to the distal region of the catheter, and
the support is configured such that, while the support expands from the contracted state to the expanded state, at least one of the segments slides distally from the proximal segment.

In an application, a distal segment is fixedly coupled to the capsule.

In an application, the plurality of coaxial segments includes at least 2 segments.

In an application, the plurality of coaxial segments includes fewer than 15 segments.

In an application, the plurality of coaxial segments includes 2-15 segments.

In an application, at least two of the segments have a radial thickness of 0.05-0.5 mm.

In an application, at least two of the segments have a length of 3-15 mm.

In an application, the support is configured such that, while the support expands from the contracted state to the expanded state, a first one of the segments that is adjacent to a second one of the segments slides distally, along the distal portion axis, between 2 mm and 14 mm with respect to the second one of the segments.

In an application:
each of the segments longitudinally overlaps with at least one other of the segments, and
while the support is in the expanded state, at least two of the segments longitudinally overlap to a lesser degree than while the support is in the contracted state.

In an application, while the support is in the expanded state, a diameter of a distal-most segment is 2-10 mm greater than a diameter of the proximal segment.

In an application, while the support is in the expanded state, a largest dimension of a distal-most segment perpendicular to the distal portion axis is 2-10 mm greater than a largest dimension perpendicular to the distal portion axis of the proximal segment.

The present invention will be more fully understood from the following detailed description of applications thereof, taken together with the drawings, in which:

### BRIEF DESCRIPTION OF THE DRAWINGS

FIGS. 1A-B are schematic illustrations showing a catheter support for use with a catheter, in accordance with some applications of the invention; and

FIGS. 2A-B are schematic illustrations showing use of a distal portion of a delivery tool, in accordance with some applications of the invention.

### DETAILED DESCRIPTION OF EMBODIMENTS

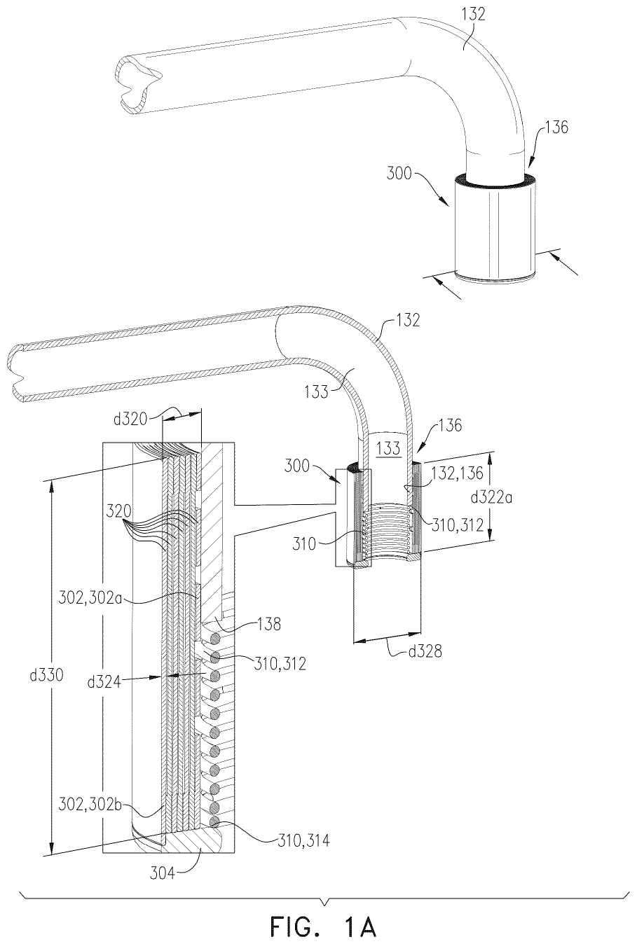

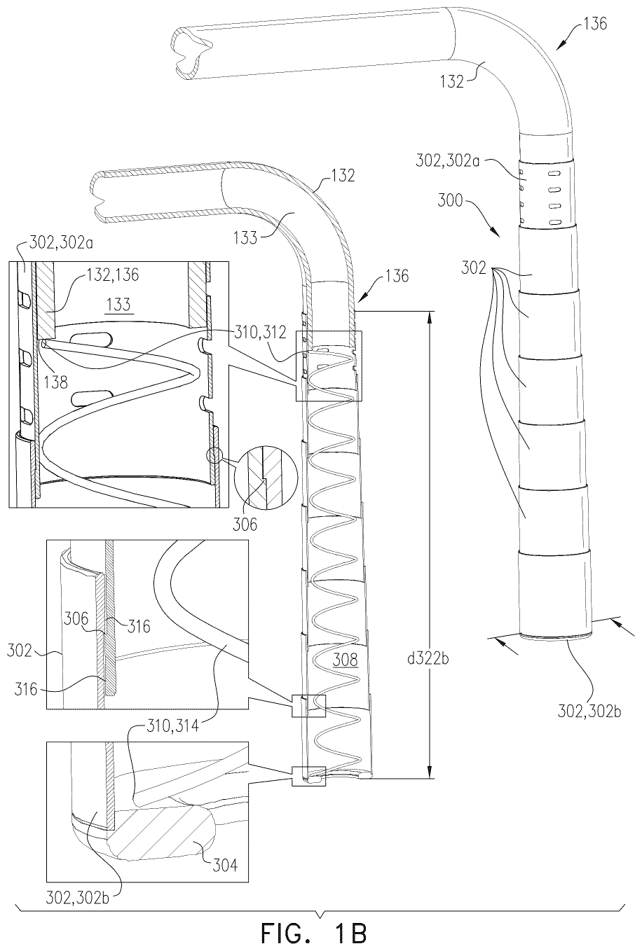

Reference is made to FIGS. 1A-B, which are schematic illustrations showing a catheter support 300 for use with a catheter 132, in accordance with some applications of the invention. Support 300 may be used with other catheters.

As shown, support 300 has a contracted state (FIG. 1A) and an expanded state (FIG. 1B). As shown in FIG. 1B, the support is shaped to define a support-lumen 308, and is typically used to mechanically support elements that are disposed within and beyond the support-lumen. For the sake of clarity, FIGS. 1A-B show support 300 without an element disposed within support-lumen 308.

Support 300 is fixedly coupled to a distal region 136 of catheter 132, and it is typically desirable for the support to have a limited profile when it is in the contracted state, in order for the contracted support to add as little bulk as possible to the contour of the catheter. For some applications, while support 300 is in the contracted state, the support has a radial thickness d320 of between 1 mm and 3 mm.

It is also typically desirable that support 300 has a limited length while it is in the contracted state. For some applications, and as shown, support 300 comprises a plurality of coaxial segments 302 that are nested with respect to each other. Typically for such applications, support 300 has at least two segments. For example, support 300 may have fewer than 15 segments, e.g. 2-15 segments. Segments 302 are typically dimensioned to each have a limited length d330 (e.g., between 4 mm and 18 mm) and a radial thickness d324 (e.g., between 0.05 mm and 0.5 mm), thereby reducing the total length and thickness of support 300 while the support is contracted.

Segments 302 typically longitudinally overlap while support 300 is contracted (FIG. 1A), further reducing the support's total length d322a when the support is contracted. Typically for such applications, support 300 is expanded by reducing the longitudinal overlap between segments 302, e.g., at least some segments 302 move longitudinally with respect to others of the segments 302 in order for support 300 to expand.

For some applications, support 300 has an expanded length d322b (FIG. 1B) that is two to five times longer than contracted length d322a. For example, support 300 may expand to become between 10 and 60 mm longer (e.g., between 10 and 50 mm longer) than contracted length d322a (FIG. 1A).

For some applications, in order for support 300 to expand, at least one of the segments slides distally from proximal segment 302a. For example, and as shown, segments 302 move distally with respect to segments having a smaller diameter. Since each progressively distal segment 302 has a larger diameter than the immediately proximal segment, support 300 tapers proximally in the expanded state. For some such applications, while the support is in the expanded state, a diameter of distal segment 302 b is between 2-10 mm greater than a diameter of proximal segment 302a.

For some applications, in the absence of an external force being applied, support 300 self-expands from the contracted state to the expanded state, e.g., due to a spring 310 disposed within support 300 being in compression when support 300 is in the contracted state. Typically for such applications, and as shown, spring 310 occupies only a portion of support-lumen 308, such that unoccupied space remains within the support-lumen for passage of elements that support 300 mechanically supports. Typically, a proximal portion 312 of the spring contacts a distal region 136 (e.g., distal end 138) of catheter 132, and a distal portion 314 of the spring contacts a distal portion 304 of the support. For some applications, and as shown in the inset of FIG. 1A, proximal segment 302a is shaped to define grooves in which a portion of spring 310 is disposed.

For some such applications, and as shown, the self-expansion of support 300 is limited, such that the support does not expand beyond the expanded length. For example, and as shown in the circular inset of FIG. 1B, each pair of segments 302 may be complimentarily shaped to define a step 316. When segments 302 slide progressively distally, each successive segment 302 is pulled distally, until respective steps 316 of each pair of segments meet each other at an interface 306, thereby inhibiting further longitudinal expansion of support 300.

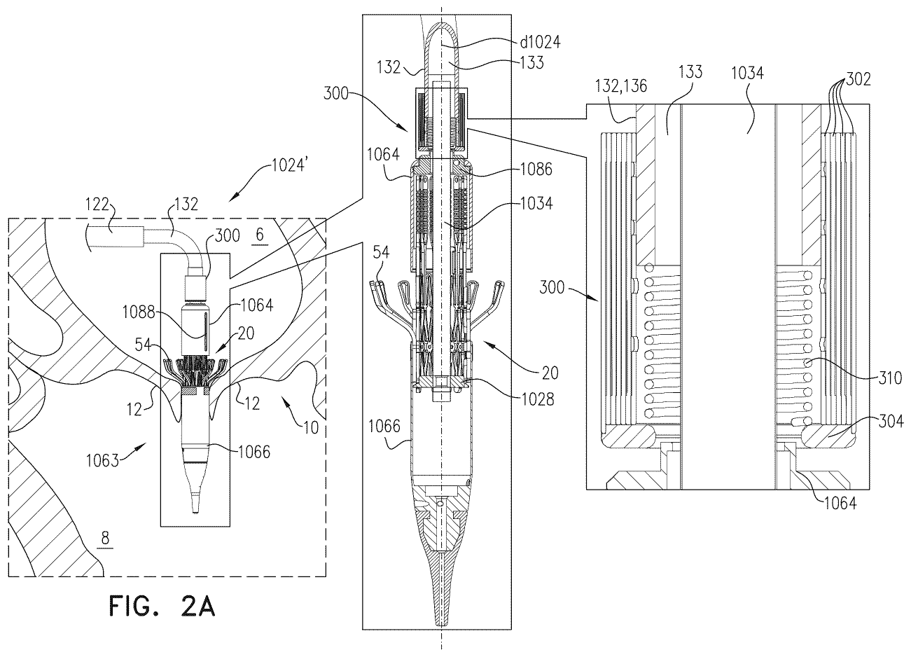

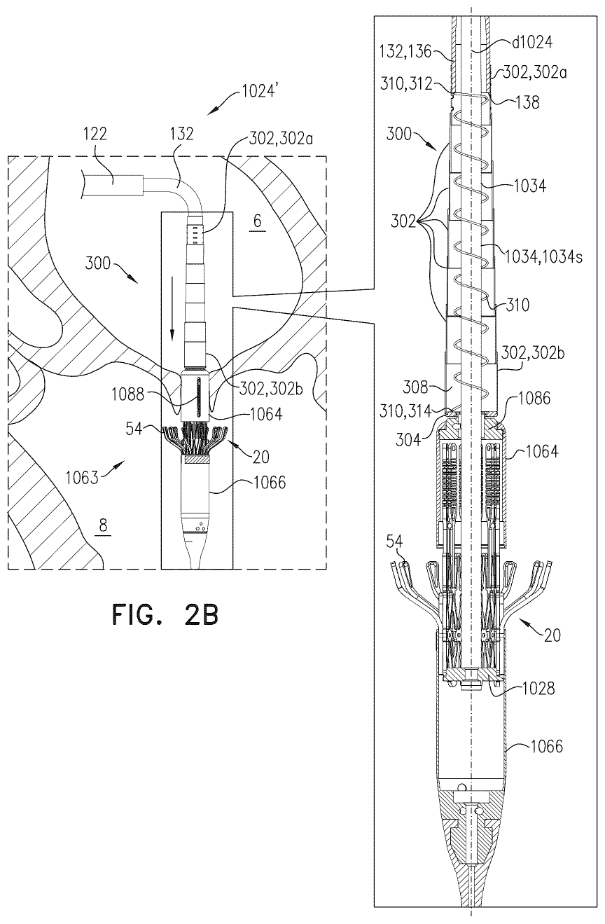

Reference is made to FIGS. 2A-B, which are schematic illustrations showing use of a distal portion 1024′ of a delivery tool, in accordance with some applications of the invention.

Distal portion 1024′ is shown in FIGS. 2A-B being used to deliver an implant 20 (e.g., a prosthetic valve) at the mitral valve. A capsule assembly 1063 is shown being delivered to the mitral valve using a first catheter 122 and a second catheter 132 of a steerable catheter system. This nonlimiting example is not meant to exclude use of other steerable catheter systems in combination with the apparatus described hereinabove with reference to FIGS. 1A-B, mutatis mutandis.

FIG. 2A shows distal portion 1024′ being used to deliver implant 20 to the mitral valve, at a stage of deployment in which a plurality of flanges 54 have been deployed within atrium 6. The cross-sectional view of FIG. 2A shows a shaft 1034 extending from within a catheter-lumen 133 of catheter 132, through support 300 and to a mount 1028. Typically, at least a portion of shaft 1034 has a greater flexibility than catheter 132. While the flexibility of shaft 1034 may be advantageous while the shaft is navigated through the confines of the vasculature, additional rigidity may be desired when advancing capsule assembly 1063 into ventricle 8. That is, the flexibility of shaft 1034 could allow mechanical forces that capsule assembly 1063 may encounter when advancing through the mitral valve to alter an orientation of the capsule assembly. As described below, support 300 is configured to reduce the likelihood that such mechanical forces would alter an orientation of capsule assembly 1063.

In the right inset of FIG. 2A, support 300 is in the contracted state, and distal portion 304 of support 300 is shown abutting a proximal capsule 1064 of capsule assembly 1063. While support 300 is in the contracted state, spring 310 is compressed, such that it applies a pushing force to proximal capsule 1064. The operator regulates the distal advancement of capsule assembly 1063, by controlling the advancement of shaft 1034, to which the capsule assembly is coupled.

FIG. 2B shows implant 20 at a stage of deployment at the mitral valve in which the capsule assembly is advanced distally, through the mitral valve, e.g., until it is determined that leaflets 12 of a native valve 10 are coapting upstream of flanges 54 during ventricular systole. As shown in FIG. 2B, distally advancing capsule assembly 1063 along a distal portion axis d1024, by distally advancing shaft 1034, allows support 300 to self-expand into the expanded state. Since support 300 has a greater stiffness than shaft 1034, the expanded support provides mechanical support to a supported portion 1034s of shaft 1034 that is disposed within support-lumen 308, thereby stabilizing the capsule assembly and/or implant 20 that are disposed distally to support-lumen 308. For some applications, and as shown, support 300 (e.g., distal portion 304 thereof) continues to extend to capsule assembly 1063 while the support is in the expanded state. For some such applications, support 300 (e.g., distal segment 302b thereof) is fixedly coupled to capsule assembly 1063 (e.g., to proximal capsule 1064).

In this way, upon suitable orienting of the capsule assembly, support 300 facilitates placement of the flanges 54 downstream of the leaflets, as shown in FIG. 2B, by reducing the effect of mechanical forces that capsule assembly 1063 may encounter when advancing through the mitral valve.

Subsequently, capsule assembly 1063 is typically retracted proximally (e.g., along distal portion axis d1024), with respect to catheter 132, until it is determined that flanges 54 have engaged leaflets 12. Typically for such applications, proximal movement of capsule assembly 1063 causes support 300 to contract at least partially from the expanded state to the contracted state, e.g., by proximal capsule 1064 pushing proximally upon distal portion 304 of the support. That is, support 300 typically does not include a locking mechanism that would prevent proximal movement of the capsule assembly from contracting the support. After capsule assembly 1063 has been partially retracted as described, proximal capsule 1064 is typically retracted proximally, in relation to shaft 1034, further exposing the implant.

For some applications, proximal capsule 1064 is proximally retracted by applying a rotational force to a proximal end of shaft 1034 or other longitudinal member that engages a disc-assembly 1086. Disc-assembly 1086 is typically fitted within proximal capsule 1064, and comprises a proximal disc that is rotationally coupled to and rotationally movable with respect to a distal disc. Further typically, the exterior of the proximal disc defines external screw threading that is complementary to internal screw threading defined by the interior proximal capsule 1064. Further typically for such applications, the proximal capsule 1064 is shaped to define one or more longitudinal tracks 1088 that traverses internal threading. Since the proximal disc is fixedly coupled to the capsule catheter, rotation of the capsule catheter with respect to shaft 1034 screws the proximal disc along internal threading of proximal capsule 1064. At the same time, the distal disc is inhibited from rotating because distal disc: (i) is fixedly coupled to shaft 1034, and (2) engages track 1088 of proximal capsule 1064. Thus, screwing of the proximal disc pushes the distal disc along track 1088, thereby translating rotational movement of the proximal disc into axial movement of proximal capsule 1064 with respect to disc-assembly 1086, as well as to mount 1028. Finally, distal capsule 1066 is advanced distally in order to further expose the implant and mount 1028, after which distal portion 1024′ is withdrawn from the subject. Further details regarding this technique are described, mutatis mutandis, with reference to FIG. 14D of US Provisional Patent Application 63/350,200 to Albitov et al., which is incorporated herein by reference.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for use at a heart of a subject, the apparatus comprising:
a delivery tool dimensioned for percutaneous delivery of an implant to the heart, the delivery tool comprising:
a catheter defining a catheter-lumen,
a shaft, at least a first portion of the shaft slidably housed within the catheter-lumen, and
a distal portion that defines a distal portion axis and comprises:
a capsule, the capsule:
configured to house at least a portion of the implant,
coupled to a distal portion of the shaft, and
slidable along the distal portion axis, with respect to the catheter, so that the capsule transitions from:
a first state in which the capsule is disposed at a first distance from a distal end of the catheter, to
a second state in which the capsule is disposed at a second distance from the distal end of the catheter,
wherein the second distance is greater than the first distance; and a support, the support:
fixedly coupled to a distal region of the catheter,
extending:
along the distal portion axis, and
from the distal end of the catheter toward the capsule, and
configured to expand, while the capsule transitions from the first state to the second state:
from a contracted state in which the support has a first length, to
an expanded state in which the support has a second length.

2. The apparatus according to claim 1, wherein a proximal region of the support is fixedly coupled to the distal region of the catheter.

3. The apparatus according to claim 1, wherein:
the support is configured to define a support-lumen,
a second portion of the shaft is disposed within the support-lumen, and
while the support is in the expanded state, the support has a stiffness that is greater than a stiffness of the second portion of the shaft.

4. The apparatus according to claim 1, wherein the support is configured such that while the capsule transitions from the first state to the second state, the support self-expands from the contracted state to the expanded state.

5. The apparatus according to claim 1, wherein:
the capsule is reversibly slidable along the distal portion axis, with respect to the catheter, such that the capsule is transitionable from the second state to the first state, and
while the capsule transitions from the second state to the first state, the support is configured to contract from the expanded state to the contracted state.

6-7. (canceled)

8. The apparatus according to claim 1, wherein, while the support is in the expanded state, the support has an expanded length that is between 10 mm and 60 mm.

9. The apparatus according to claim 1, wherein:
while the support is in the contracted state, the support has a contracted length, and
the expanded length that is 10-50 mm longer than the contracted length.

10. The apparatus according to claim 8, wherein:
while the support is in the contracted state, the support has a contracted length, and
the expanded length is 2-5 times longer than the contracted length.

11. The apparatus according to claim 1, wherein the support is configured such that while the support is in the expanded state, the support extends from the distal end of the catheter to the capsule.

12. The apparatus according to claim 11, wherein the support is configured such that while the support expands from the contracted state to the expanded state, the support extends from the distal end of the catheter to the capsule.

13. The apparatus according to claim 1, wherein:
the support comprises a plurality of coaxial segments,
a proximal one of the coaxial segments is coupled to the distal region of the catheter, and
the support is configured such that, while the support expands from the contracted state to the expanded state, at least one of the segments slides distally from the proximal segment.

14. The apparatus according to claim 13, wherein a distal segment is fixedly coupled to the capsule.

15. The apparatus according to claim 13, wherein the plurality of coaxial segments comprises at least 2 segments.

16. (canceled)

17. The apparatus according to claim 13, wherein the plurality of coaxial segments comprises 2-15 segments.

18. The apparatus according to claim 13, wherein at least two of the segments have a radial thickness of 0.05-0.5 mm.

19. The apparatus according to claim 13, wherein at least two of the segments have a length of 3-15 mm.

20. The apparatus according to claim 13, wherein, the support is configured such that, while the support expands from the contracted state to the expanded state, a first one of the segments that is adjacent to a second one of the segments slides distally, along the distal portion axis, between 2 mm and 14 mm with respect to the second one of the segments.

21. The apparatus according to claim 13, wherein:
each of the segments longitudinally overlaps with at least one other of the segments, and
while the support is in the expanded state, at least two of the segments longitudinally overlap to a lesser degree than while the support is in the contracted state.

22. The apparatus according to claim 21, wherein, while the support is in the expanded state, a diameter of a distal-most segment is 2-10 mm greater than a diameter of the proximal segment.

23. The apparatus according to claim 21, wherein, while the support is in the expanded state, a largest dimension of a distal-most segment perpendicular to the distal portion axis is 2-10 mm greater than a largest dimension perpendicular to the distal portion axis of the proximal segment.
